(19) United States
(12) Patent Application Publication
(10) Pub. No.: US 2025/0090056 A1
(43) Pub. Date: Mar. 20, 2025

(54) Mental Health and Addiction Bracelet

(71) Applicant: Kristin Thames, Calabasas, CA (US)

(72) Inventor: Kristin Thames, Calabasas, CA (US)

(21) Appl. No.: 18/812,967

(22) Filed: Aug. 22, 2024

Related U.S. Application Data
(60) Provisional application No. 63/534,023, filed on Aug. 22, 2023.

(51) Int. Cl. A61B 5/1455; A61B 5/00; A61B 5/11

(52) U.S. Cl. CPC A61B 5/14552; A61B 5/1112; A61B 5/681; A61B 5/7435; A61B 5/746; A61B 5/7475

(57) ABSTRACT

A mental health and addiction bracelet which monitors the user's pulse and blood oxygenation levels and automatically transmits an alert to a response team if the blood oxygenation level falls below a specific percentage or when the pulse rate falls below a certain level. The bracelet also includes a location tracking function and a locking mechanism to prevent the user from removing the bracelet without authorization.

### CROSS-REFERENCE TO RELATED APPLICATIONS

This application claims priority from U.S. Provisional Application 63/534,023, filed Aug. 22, 2023, and incorporates the filing in its entirety.

### FIELD OF INVENTION

The present invention relates to a mental health and addiction bracelet which monitors a user's pulse rate and blood oxygenation levels and automatically transmits an alert to a response team when a sensor determines when the blood oxygenation level falls below a pre-determined threshold or when the pulse rate falls below a pre-determined level. The bracelet also includes a location tracking function and a locking mechanism to prevent the wearer from removing the bracelet without authorization.

### BACKGROUND OF THE INVENTION

Many people require greater care as the age, whether due to physical health constraints, dementia, or other mental health issues. It can be challenging and overwhelming to provide surveillance for each hour of the day, particularly for a caregiver who is a family member or friend and typically untrained in providing such care. People with dementia and other mental health issues sometimes wander off from their caregiver, become disoriented, and sometimes get lost and have no means to contact the caretaker of their whereabouts. In other situations, people with mental health issues can become depressed and have suicidal thoughts. Individuals with drug addictions, particularly opioid addiction, represent another growing group in the population who require continued monitoring. In many situations, it is too expensive to hire a subscription company to monitor an individual 24/7 but impractical if not impossible for a family member or friend to provide continued care and monitoring. Addiction is both a physical condition and a mental condition. In many instances, it can be easier to emotionally to ask for and receive help from a third party rather than a family member or close friend.

### PRIOR ART

The prior art is replete with devices designed to assist both caregivers and law enforcement. For example, tracking or location bracelets and anklets, commonly referred to as ankle monitors which cannot be removed by the wearer, are well known in determining a person's location. Further, the creation of smart watches with GPS location services have facilitated determining a person's location. Some smart watches have been designed that monitor pulse rate and even blood oxygenation. Other devices also have been developed which can be worn continuously to monitor an individual's vital signs. For example, U.S. Pat. No. 9,318,013 B2 to Zobar, issued Apr. 19, 2016, is directed to a drowsiness warning system in the form of an alarm bracelet which monitors the wearer's pulse rate and activates a vibration mode when the pulse rate falls below a calculated predetermined level. The vibration is intended to alert the wearer and not a response team. Further, the bracelet does not monitor blood oxygen levels, does not have a user alert button, and does not prevent the user from removing the device.

Both U.S. Patent Application Publication 2018/0110923 A1 to Kaplan et al., published Apr. 26, 2018 and U.S. Patent Application Publication 2021/0128078 A1 to Kiani et al., published May 6, 2021, provide wearable devices for monitoring blood oxygen levels to prevent an opioid overdose for a wearer. In Kiani et al., a monitoring service notifies a first set of contacts while in Kaplan et al., the system sounds an alarm and automatically injects an opioid antidote. Neither Kiani et al. or Kaplan et al. offers the wearer of the device with an alert button.

Despite the advances of the prior art, a need still exists for a single wearable device that can monitor the combination of pulse rate and blood oxygenation levels and send an alert in response to either the pulse rate or the blood oxygenation level falling below a pre-determined threshold to a pre-selected response team. Such a device also should include a GPS or similar tracking location means to facilitate locating the wearer after an alert has been received. And, such a device also should comprise a locking mechanism to prevent the wearer from removing the device without authorization. Further, such a device should include a user-activated alert means that can be activated by the wearer when immediate assistance is desired.

### SUMMARY OF THE INVENTION

The invention of the present subject matter relates to a mental health and addiction bracelet which monitors the user's pulse rate and blood oxygenation levels and automatically alerts a response team when the user's pulse rate or blood oxygenation level falls below a predetermined level. The bracelet of the present subject matter includes a GPS or location tracking device, a display face having an alert button disposed on the side thereof and cannot be removed without authorization.

Accordingly, it is an object of the present subject matter to provide a mental health and addiction bracelet which monitors the user's pulse rate and blood oxygenation levels and automatically transmits an alert to a response team when the user's pulse rate or blood oxygenation level falls below a pre-determined level.

It is another object of the present subject matter to provide a mental health and addiction bracelet which transmits an alert to a response team which is a subscription company, such as a Narcan Response Team.

It is still another object of the present subject matter to provide a mental health and addiction bracelet which automatically transmits an alert to a response team when the user's pulse rate falls below a pre-determined level of 50 beats/minute.

It is a yet another object of the present subject matter to provide a mental health and addiction bracelet which automatically transmits an alert to a response team when the user's blood oxygenation level falls below a pre-determined level of 90.0%.

It is a further object of the present subject matter to provide a mental health and addiction bracelet which has a locking mechanism such that the bracelet is not removable from the user's wrist without authorization.

It is yet a further object of the present subject matter to provide a mental health and addiction bracelet comprising a GPS tracking device or other such location traction device to determine the user's location, particularly after an alert has been transmitted to a response team.

It is still a further object of the present subject matter to provide a mental health and addiction bracelet capable of alerting a response team composed of people chosen by the user.

It is an additional object of the present subject matter to provide a mental health and addiction bracelet having a display face with an interface screen and a user-activated alert button disposed on the side of the display face.

It is an additional object of the present subject matter to provide a mental health and addiction bracelet for a person have drug addictions such as an opioid addiction.

It is another object of the present subject matter to provide a mental health and addiction bracelet for a person having a drug addiction which monitors the user's pulse rate and blood oxygenation levels and automatically transmits an alert to a Narcan Response Team when the user's pulse rate or blood oxygenation level falls below a pre-determined level.

It is still another object of the present subject matter to provide a mental health and addiction bracelet having a user-activated alert button that the wearer can initiate to transmit an alert to a response team when the user is lost or suicidal.

These and other objects of the present subject matter are accomplished by providing a mental health and addiction bracelet having a band to be worn about the wrist of a user and which cannot be removed without authorization, the mental health and addiction bracelet comprising a pulse oximeter for measuring the user's pulse rate and blood oxygenation levels, a sensor for monitoring the user's pulse rate and blood oxygenation levels and transmitting an alert to a response team when the user's pulse rate or blood oxygenation level falls below a pre-determined level, a location tracking device, a display face having a user interface screen, and a user-activated alert button disposed on the side of the display face which can be initiated to transmit an alert by the wearer.

### DETAILED DESCRIPTION OF THE INVENTION

In the following description, numerous specific details are set forth to clearly describe various specific embodiments disclosed herein. One skilled in the art, however, will understand that the presently claimed invention may be practiced without all the specific details discussed below. In other instances, well known features have not been described so as not to obscure the invention.

It is to be understood that the term “bracelet” and similar terms may be used throughout this specification and it is to be understood that such terms are interchangeable and all refer to mental health and addiction bracelet of the present subject matter.

Further, it is to be understood that the phraseology and terminology used herein is for the purpose of description and should not be regarded as limiting. The use of the terms “including”, “comprising”, “having”, and variations thereof herein is meant to encompass the items listed thereafter and equivalents thereof as well as additional items.

The present subject matter is directed a mental health and addiction bracelet having a band which is to be worn about the wrist of a user and which has a locking mechanism to prevent the wearer from removing the bracelet without authorization. The bracelet comprises a pulse oximeter for measuring the wearer's pulse rate and blood oxygenation levels, a sensor for monitoring the wearer's pulse rate and blood oxygenation level and for transmitting an alert to a response team when either the wearer's pulse rate or the wearer's blood oxygenation level falls below a pre-determined threshold. In the preferred embodiment, the sensor will send an alert when the wearer's pulse rate is below 50 beats/minute or when the wearer's blood oxygenation level is below 90%.

Because different individuals have different pulse rates, the threshold pulse for sending an alert may be adjusted by a medical provider or other authorized person to be more accurate for the particular wearer of the medical health and addiction bracelet of the present subject matter. It is contemplated to be within the scope of the present invention that the bracelet also can be programmed to send an alert in response to other conditions measured by the pulse oximeter, for example, a very high pulse rate. It further will be obvious to those skilled in the art that the bracelet can be programmed to automatically transmit an alert when the wearer leaves a predefined area or wanders into a dangerous area.

The location tracking function may be a GPS function, may be served via triangulation of position relative to cellular technology relay towers, Bluetooth-based positioning (which utilizes local IP addresses to determine the location of a device, similar to using cellular towers) or any equivalent location providing technology.

In one embodiment, the medical health and addiction bracelet of the present subject matter may be made to appear intentionally obvious that it is not removable, thereby notifying everyone that the wearer cannot remove the bracelet and therefore it cannot be stolen from them. In an alternate embodiment, the bracelet may be made to appear visually interchangeable with existing smart watches, thereby preventing social embarrassment for the wearer and those around the wearer. In both embodiments, the user-activated alert button on either side of the display face in a manner similar to that of an actual watch button or displayed on one side of the display face itself.

The mental health and addiction bracelet of the present subject matter is provided to save lives. For individuals having mental health issues, particularly severe depression and feelings of suicide, the user-activated alert button offers a way for the wearer to alert a response team if the user is feeling suicidal. Too often, confessing to suicidal feelings to a family member or friend can be difficult as the suicidal individual does not want to burden family and friends. The use of a third-party suicide prevention response team reduces the barrier of embarrassment and may promote the individual to seek help. However, the response team can be selected by the wearer when the response is from an alert transmitted from the user-activated alert button.

Opioid addiction is rampant. Opioids can induce respiratory depression by invoking a centrally mediated decrease in involuntary respiratory rate, which in severe cases can cause a decrease in oxygen saturation. If respiratory depression is opioid induced, both low respiratory rate and low oxygen saturation will be present. Thus, the monitoring of blood oxygenation levels and pulse rates by the medical health and addiction bracelet of the present subject matter can be a significant tool in monitoring a drug addict, particularly an opioid addict. In this type of situation, when the wearer's pulse rate and/or blood oxygenation level falls below the pre-determined threshold, an alert is immediately and automatically transmitted to a subscription company, particularly a Narcan Response Team. The response team then can access the location tracking information to get to the wearer and administer Narcan if necessary or call 911, depending upon the circumstances.

The medical health and addiction bracelet also may be used as part of a court-ordered drug rehabilitation. It is less resource intensive to monitor someone remotely than to keep them in an inpatient treatment facility, and there is an in-between state of recovery in which the bracelet would be a more appropriate continuous intervention, especially for those who do not deal well with the atmosphere of an inpatient treatment facility. Although it is unknown how common it is, the stress of being in an inpatient facility may be detrimental to some individuals' recovery process. The bracelet also may be used for patients located at an inpatient treatment facility to assist staff in rapidly locating patients who may wander off from a predefined area.

The medical health and addiction bracelet of the present subject matter also is suitable for use for individuals with other mental health conditions which prevent the wearer from being willing or capable of seeking assistance and help when needed, such as individuals with dementia who are at high risk of falling or forgetting the year or where they are located. In one embodiment, the bracelet may include an “I'm confused” or “I'm lost” button.

The response team can be from a list of individuals selected by the wearer, including for example, family members, friends, therapist, and other mental health providers, or can be a subscription company. However, to whom the alerts are transmitted is determined by authorized personnel and cannot be changed by the wearer. Offering the option of transmitting an alert to a list of individuals rather than a subscription company provides some of the benefits of the bracelet of the present subject matter to those who do not have funds of insurance to pay for a subscription service or those who are not located in an are in which such a company operates. As will be obvious to those skilled in the art, the subscription to a company also may be provided as a government service, as part of an addiction recovery program or an eldercare program.

While several illustrative embodiments of the invention have been shown and described, numerous variations and alternative embodiments will occur to those skilled in the art. Such variations and alternative embodiments are contemplated and can be made without departing from the scope of the invention as defined in the appended claims.

The above detailed description of exemplary and preferred embodiments is presented for the purposes of illustration and disclosure in accordance with the requirements of the law. It is intended to be exemplary but not exhaustive, and is not intended to limit the invention to the precise forms described, but only to enable others skilled in the art to understand how the invention may be suited for a particular use of implementation. Applicant has made this disclosure with respect to the current state of the art, but also contemplates advancements and that adaptations in the future may take into consideration those advancements in accordance with the then current state of the art. It is intended that the scope of the invention be defined by the Claims as written and equivalents as applicable. Reference to a claim element in the singular is not intended to mean “one and only one” unless explicitly so stated. No claim element herein is intended to be construed under the provisions of 35 U.S.C. 112(f), unless the element is expressly recited using the exact phrase “means for . . . ” and no method or process step herein is to be construed under the provisions of 35 U.S.C. section 112(f) unless the step, or steps, are expressly recited using the exact phrase “step(s) for . . . ”.

1. A mental health and addiction bracelet having a band to be worn about the wrist of a user comprising:
a. a pulse oximeter for measuring the user's pulse rate and blood oxygenation levels;
b. a sensor for monitoring the user's pulse rate and blood oxygenation levels and transmitting an alert to a response team when the user's pulse rate falls below a threshold level or when the user's blood oxygenation level falls below a threshold level;
c. a location tracking function for determining the location of the user;
d. a display face having user interface screen on said band;
e. a user-activated alert button disposed on the side of said display face, and
f. a locking mechanism to prevent the user from removing the bracelet without authorization.

2. The mental health and addiction bracelet in accordance with claim 1, wherein an alert is sent to a response team when the user's pulse rate falls below 50.

3. The mental health and addiction bracelet in accordance with claim 1, wherein an alert is sent to a response team when the user's blood oxygenation level falls below 90%.

4. The mental health and addiction bracelet in accordance with claim 1, wherein when an alert is sent to a response team, said response team can determine the location of the user by accessing by location tracking function.

5. The mental health and addiction bracelet in accordance with claim 1, wherein said user-activated alert button can be initiated by said user to transmit an alert to a response team.
